(19) United States
(12) Patent Application Publication
(10) Pub. No.: US 2008/0070846 A1
(43) Pub. Date: Mar. 20, 2008

(54) TREATING ACUTE EXACERBATIONS OF ASTHMA USING A KETOLIDE

(75) Inventors: Richard B. Nieman, Princeton, NJ (US); Bruno Leroy, Saint Nom la Breteche (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Aventis Pharmaceuticals Inc

(21) Appl. No.: 11/753,652

(22) Filed: May 25, 2007

Related U.S. Application Data
(63) Continuation of application No. PCT/US05/43277, filed on Nov. 29, 2005.
(60) Provisional application No. 60/631,812, filed on Nov. 30, 2004.

(51) Int. Cl. A61K 31/7048; A61P 11/06; A61P 31/04

(52) U.S. Cl. 514/29

(57) ABSTRACT

Provided herein is a method of treating a patient suffering from, or subject to, acute asthma exacerbations comprising administering to the patient a pharmaceutically effective amount of a ketolide.

This application claims the benefit of U.S. provisional application No. 60/631,82, filed Nov. 30, 2004.

### FIELD OF THE INVENTION

This invention is directed to the use of a ketolide for treating acute asthma exacerbations in a patient.

### BACKGROUND OF THE INVENTION

Acute exacerbations of asthma are an important healthcare problem, and accounted for 1.8 million visits to the emergency department, 465,000 hospitalizations, and 4,487 deaths in the USA in 2000. [CDC, National Center for Health Statistics, see “Asthma Prevalence, Health Care Use and Mortality, 2002,”accessed Nov. 9, 2005, at http://www.cdc.gov/nchs/products/pubs/pubd/hestats/asthma/asthma.htm]. Furthermore, increased disease burden and asthma symptoms frequently persist for at least one month after emergency department discharge following an asthma exacerbation. [see Lenhardt R., Walter J. J., McDermott M. F, et al., Burden of Asthma Persists One Month after Emergency Department Discharge: Results from the Illinois Emergency Department Asthma Collaborative (IEDAC). Acad. Emerg. Med 2004; 11: No. 5 534].

Current treatment strategies for acute exacerbations of asthma rely heavily on bronchodilators and inhaled or systemic corticosteroids. Asthma guidelines recommend regular inhaled corticosteroid treatment for patients with persistent symptoms, and doubling the dose of inhaled corticosteroids is widely used in cases where asthma control has deteriorated. [see British Thoracic Society/Scottish Intercollegiate Guidelines Network. British Guideline on the Management of Asthma. Thorax 2003; 58 (Suppl. 1): i1-i94]. Two recently published randomized, controlled trials, however, did not show any evidence of improved outcome with doubling doses of inhaled corticosteroid at the onset of exacerbation. [see Harrison T. W., Oborne J., Newton S., Tattersfield A. E. Doubling the Dose of Inhaled Corticosteroid to Prevent Asthma Exacerbations: Randomised Controlled Trial. Lancet 2004; 363: 271-5; and see also FitzGerald J. M., Becker A., Sears M. R., et al. Doubling the Dose of Bbudesonide versus Maintenance Treatment in Asthma Exacerbations. Thorax 2004; 59:550-6].

Although a course of oral steroids is standard treatment practice in most countries, it is remarkable that there are no published controlled studies comparing oral steroids with placebo in the treatment of asthma exacerbations. However, several controlled studies have compared the effects of short-course oral or parenteral corticosteroids with an alternative active treatment in place of placebo in the treatment of acute asthma exacerbations. In a pediatric study (mean age 8.0 years), treatment with oral prednisone resulted in mean increases in PFTs after 7 days as follows: PEF, 78 L/min; FEV₁, 0.37 L; FVC, 0.45 L; and FEF₂₅₋₇₅%, 0.41 L/sec [see Manjra A. I., Price J., Lenney W., Hughes S., Barnacle H. Efficacy of Nebulized Fluticasone Propionate Compared with Oral Prednisolone in Children with an Acute Exacerbation of Asthma. Respir. Med. 2000; 94: 1206-14]. In adult patients, PEF improved by 46 L/min after seven to ten days of oral prednisone [see Schuckman H., DeJulius D. P., Blanda M., Gerson L. W., DeJulius A. J., Rajaratnam M. Comparison of Intramuscular Triamcinolone and Oral Prednisone in the Outpatient Treatment of Acute Asthma: A Randomized Controlled Trial. Ann. Emerg. Med. 1998; 31:(3) 333-8]. A tapering course of oral prednisone in adults with mild asthma exacerbations resulted in improvements in PEF % predicted from 73% at onset of treatment to 85% after 16 days. [see Levy M. L., Stevenson C., Maslen T. Comparison of Short Courses of Oral Prednisolone and Fluticasone Propionate in the Treatment of Adults with Acute Exacerbations of Asthma in Primary Care. Thorax 1996; 51: 1087-92]. A study of oral prednisone following an acute asthma exacerbation in adults showed that FEV₁ and PEF, measured after bronchodilator use, increased by 0.55 L and 77 L/min, respectively, over seven to ten days. [see FitzGerald J. M., Shragge D., Haddon J., et al. A Randomized, Controlled Trial of High Dose, Inhaled Budesonide versus Oral Prednisone in Patients Discharged from the Emergency Department Following an Acute Asthma Exacerbation. Can. Respir. J. 2000; 7 (1):61-67].

Beyond the usage of bronchodilators and inhaled or systemic corticosteroids to treat acute exacerbations of asthma, antibiotics are often prescribed for acute exacerbations of asthma. However, guidelines recommend against prescribing antibiotics in this setting. [see British Thoracic Society/Scottish Intercollegiate Guidelines Network. British Guideline on the Management of Asthma. Thorax 2003; 58 (Suppl. 1): i1-i94; see also pages 63-70 of NAEPP Expert Panel Report: Guidelines for the Diagnosis and Management of Asthma, Update on Selected Topics 2002, accessed Nov. 15, 2005 (http.//www.nh/bi.nih.gov/guidelines/asthma/asthmafullrpt.pdf]; and see also Henderson M., Rubin E., Misuse of Antimicrobials in Children with Asthma and Bronchiolitis: A Review. Pediatr. Infect. Dis. J. 2001; 20:214-215]. Furthermore, NAEPP guidelines state that evidence needs to be obtained to address the whether antibiotics should be used in the treatment of acute exacerbations of asthma.

There are controlled studies that investigated whether antibiotics have a potential role in the treatment of acute asthma exacerbations. Despite clinical data showing that the macrolide clarithromycin can reduce the severity of bronchial hyperresponsiveness in asthmatic patients, [see E. Kostadima, S. Tsiodras, E. I. Alexopoulos, et al., Clarithromycin Reduces the Severity of Bronchial Hyperresponsiveness in Patients with Asthma. Eur. Respir, J. 2004; 23:714-7], controlled studies of macrolides for the Treatment of chronic asthma have only shown small benefits with roxithromycin and clarithromycin, and improvement was only demonstrated in some of the endpoints. [see P. N. Black, F. Blasi, C. R. Jenkins, et al., Trial of Roxithromycin in Subjects with Asthma and Serological Evidence of Infection with Chlamydia pneumoniae. Am. J. Respir. Crit. Care Med 2001; 164:536-41; and see also M. Kraft, G. H. Cassell, J. Pak, R. J. Martin, Mycoplasma pneumoniae and Chlamydia pneumoniae in Asthma*: Effect of Clarithromycin. Chest 2002; 121:1782-8]. The Cochrane Review identified only two previous placebo-controlled studies of antibiotics in acute asthma, neither of which demonstrated any benefit associated with antibiotic use [see Graham V, Lasserson T J, Rowe B H. Antibiotics for acute asthma. The Cochrane Database of Systematic Reviews 2001, Issue 2. Art. No.: CD002741. DOI: 10.1002/14651858.CD002741]. Both studies enrolled only small numbers of hospitalized patients, and most showed no sign of bacterial infection. Both studies used antimicrobial therapy but did not cover atypical bacteria such as Chlamydia or Mycoplasma pneumonia.

There is also a specific suggestion that there may be an association between acute asthma exacerbations and infection with reactivation of the atypical bacteria Chlamydia pneumoniae and Mycoplasma pneumoniae. The majority of studies however, investigating such a link have been uncontrolled and have provided conflicting evidence. [see Betsou F., Sueur J. M., Orfilla J. Anti-Chlamydia pneumoniae heat shock protein 10 antibodies in asthmatic adults, FEMS Immunol. Med. Microbiol. 2003; 35: 107-11; see also, P. A. B. Wark, S. L. Johnston, J. L. Simpson, M. J. Hensley , P. G Gibson, Chlamydia pneumoniae Immunoglobulin A Reactivation and Airway Inflammation in Acute Asthma. Eur. Respir. J. 2002; 20: 834-40; see also Leaver R., Weinberg E. G. Is Mycoplasma pneumoniae a Precipitating Factor in Acute Severe Asthma in Children? S. Afr. Med. J. 1985; 68:78-9; Lieberman D., Lieberman D., Printz S., et al. Atypical Pathogen Infection in Adults with Acute Exacerbation of Bronchial Asthma. Am. J. Respir. Crit. Care Med. 2003; 167:406-410; see also Esposito S., Droghetti R., Bosis S., Claut L., Marchioso P., Principi N. Cytokine Secretion in Children with Acute Mycoplasma pneumoniae Infection and Wheeze. Pediatr. Pulmonol. 2002; 34:122-7; and see also Thumerelle C., Deschildre A., Bouquillon C., et al. Role of Viruses and Atypical Bacteria in Exacerbations of Asthma in Hospitalized Children: A Prospective Study in the Nord-Pas de Calais Region (France). Pediatr. Pulmonol. 2003; 35:75-82].

Ketolides are a new class of antibiotics that, although structurally related to macrolides, [see Ackermann G., Rodloff A. C. Drugs of the 21st Century: Telithromycin (HMR 3647)-the First Ketolide, J. Antimicrob. Chemother. 2003; 51: 497-511], are bactericidal against C. pneumoniae and M. pneumoniae. [see Hammerschlag M. R., Roblin P. M., Bébéar C. M. Activity of Telithromycin, a New Ketolide Antibacterial, Against Atypical and Intracellular Respiratory Tract Pathogens. J. Antimicrob. Chemother. 2001; 48: Topic T1, 25-31; see also Yamaguchi T., Hirakata Y., Izumikawa K., et al. In vitro Activity of Telithromycin (HMR 3647), a New Ketolide, Against Clinical Isolates of Mycoplasma pneumoniae in Japan. Antimicrob. Agents Chemother. 2000; 44, No. 5: 1381-1382; and see also Roblin P. M., Hammerschlag M. R. In vitro Activity of a New Ketolide Antibiotic, HMR 3647, Against Chlamydia pneumoniae. Antimicrob. Agents Chemother. 1998; 42:1515-1516]. Telithromycin, 800 mg once daily, for ten days, is presently approved for treating community-acquired pneumonia. In an in vitro model, telithromycin treatment was also shown to reduce culture and PCR positivity in the lungs of mice with acute C. pneumoniae infection. [see Törmäkangas L., Saario E., David D. Bem, Bryskier A., Leinonen M., Saikku P. Treatment of Acute Chlamydia pneumoniae Infection with Telithromycin in C57BL/6J Mice. J. Antimicrob. Chemother. 2004; 53: 1101-1104]. The ketolide telithromycin, like certain macrolides, has also been shown to have immunomodulatory effects in vitro and in in vivo models. [see Araujo F. G., Slifer T. L., Remington J. S. Inhibition of Secretion of Interleukin-1α and Tumor Necrosis Factor Alpha by the Ketolide Antibiotic Telithromycin. Antimicrob. Agents Chemother. 2002; 46, No. 10: 3327-3330; and see also Nicolau D. P., Tessier P. R., Rubenstein I., Nightingale C. H. In vivo Immunomodulator Profile of Telithromycin in a Murine Infection Model. Clin. Microbiol. Infect. 2003; 9 (Suppl. 1): 397].

Accordingly, the combined properties of telithromycin having both bactericidal and immunomodulatory effects made it a good choice for a study regarding the effects of the antibiotic in the treatment of acute exacerbations of asthma. In fact, telithromycin was noted to be part of a multinational study (double-blind, randomized and placebo-controlled) to determine whether a ten-day course of telithromycin, compared with placebo, added to standard of care therapy, improves symptoms and pulmonary function tests in patients with acute exacerbations of asthma wherein the patient had no clinically obvious need for antibiotic treatment. What is needed is an effective method for treating acute asthma exacerbations using a ketolide.

### SUMMARY OF THE INVENTION

Accordingly, the present invention extends to a method of treating a patient suffering from, or subject to, acute asthma exacerbations comprising administering to the patient a pharmaceutically effective amount of a ketolide.

Moreover, in a method of the present invention, the treating, may further comprise administering a pharmaceutically effective amount of at least one an additional therapeutic agent selected from the group consisting of an inhaled corticosteroid, oral corticosteroid, bronchodilator, such as a beta-agonist, and leukotriene antagonist.

The present invention further extends to a method wherein the treating is effected in part through bactericidal activity, immunomodulatory activity, and/or anti-inflammatory activity of the ketolide.

### BRIEF DESCRIPTION OF THE DRAWINGS

The present invention will be better appreciated by reference to the following drawings.

FIG. 1 shows mean SE percent reduction in symptom severity from baseline in patients treated with telithromycin 800 mg, once daily for ten days (N=126) or placebo (N=129).

FIG. 2 shows a Kaplan-Meier Analysis of time to 50% reduction from baseline in asthma symptoms in patients treated with Telithromycin 800 mg once daily for ten days (N=126) or Placebo (N=129).

FIG. 3 shows mean SE change from baseline FEV₁ at each study visit in patients treated with Telithromycin 800 mg once daily for ten days (N=126) or Placebo (N=129).

### DETAILED DESCRIPTION OF THE INVENTION

The present invention will be better appreciated by reference to the following Detailed Description.

### Embodiments

One embodiment of the invention is directed to the method of treating wherein the ketolide is telithromycin.

Another embodiment of the invention is directed to the method of treating wherein the administration is by an oral, intravenous or inhalational route of administration. A more particular embodiment according to the invention is where the administering effected orally.

Yet another embodiment of the invention is directed to the method of treating effected in part through bactericidal activity is against C. pneumoniae and M. pneumoniae.

Another embodiment of the invention is administration of telithromycin within 24 hours of an exacerbation.

Definitions

As used above, and throughout the description of the invention including the appended claims, the following abbreviations and terms, unless otherwise indicated, are understood to have the following meanings:

FEV₁ Forced expiratory volume in 1 second

SE Standard error

TEAE Treatment-emergent adverse event

FEF_{25-75%}, Forced mid-expiratory flow rate

FVC Forced vital capacity

PEF Peak expiratory flow

SD standard deviation

PFT Pulmonary function test

LS Least square

ANOVA Analysis of variance

ANCOVA Analysis of covariance

ITT Intent-to-Treat

CDC Centers for Disease Control and Prevention

L Liter(s)

“Treating” or “treatment” means prevention, partial alleviation, or cure of the disease.

“Patient” includes humans, both male and female, ranging from 18-55 years of age.

“Antibacterial” refers to a substance that destroys bacteria or suppresses their growth or reproduction.

“Bactericidal” refers to an agent that is capable of killing bacteria.

“Bioavailable” refers to the degree to which or rate at which a drug or other substance is absorbed or becomes available at the site of physiological activity after administration.

“Immunomodulatory” refers to an agent is capable of having a particular effect of modifying or regulating one or more immune functions.

“Immune” means not susceptible or responsive. Especially, having a high degree of resistance to a disease.

“Effective amount” is meant to describe an amount of a compound effective in producing the desired therapeutic effect.

“Treatment-emergent adverse event” refers to an adverse event or reaction that occurs during the study treatment phase

Actual dosage levels of active ingredient(s) in the compositions of the invention may be varied so as to obtain an amount of active ingredient(s) that is (are) effective to obtain a desired therapeutic response for a particular composition and method of administration for a patient. A selected dosage level for any particular patient therefore depends upon a variety of factors including the desired therapeutic effect, on the route of administration, on the desired duration of treatment, the etiology and severity of the disease, the patient's condition, weight, sex, diet and age, the type and potency of each active ingredient, rates of absorption, metabolism and/or excretion and other factors.

The dose administered of a composition in accordance with the present invention is from about 200 mg/day to about 1600 mg/day. More particularly, the present invention, is administered at about 800 mg/day. The exact dosage to be used, however, will be determined based on the age and disease status of an individual patient by a skilled physician.

A composition according to the invention is preferably produced and administered in dosage units, with each unit containing, as the active constituent, a particular dose of the compound.

The dosing regimen can be rationally modified over the course of therapy so that the lowest amounts of each of the pharmaceutically effective amount of compounds used in combination which together exhibit satisfactory pharmaceutical effectiveness are administered, and so that administration of such pharmaceutically effective amount of compounds in combination is continued only SO long as is necessary to successfully treat the patient.

The regimen of a composition in accordance with the present invention provides for that which is appropriate for a particular patient, including once a day administration.

In practice, the compound of the present invention is administered in a suitable formulation to patients. It will be appreciated that the preferred route can be varied depending on the site of the condition for which administration is directed. In practice, the method of administering the compound of the present invention in a pharmaceutically acceptable dosage form to humans may include enteral, parenteral or topical administration, such as oral, intravenous or inhalational. Appropriate dosage forms for enteral administration of the compound of the present invention may include tablets, capsules or liquids. Appropriate dosage forms for parenteral administration may include intravenous administration. Appropriate dosage forms for topical administration may include nasal sprays, metered dose inhalers, dry-powder inhalers or by nebulization. It will be appreciated that the preferred route and dosage form may vary with for example the condition of the recipient. For any route of administration, divided or single doses may be used to administer the compound of the present invention.

Compositions of the present invention can further include additional pharmaceutically acceptable carriers, adjuvants, and/or biologically active substances. Compositions of the present invention, as described above, can be used in methods for treatment of acute asthma exacerbations, particularly in humans. The methods involve administering to a mammal an amount of the compositions effective to prevent, eliminate, or control the exacerbations.

Such administration may be used together with other forms of therapy, including methods involving administration of different biologically active agents to the subject. Such therapies may improve efficacy and decrease the risk of side effects compared with increasing the dose of a single agent. Ketolides can be combined with inhaled corticosteroids, for example beclomethasone, budesonide, fluticasone, or mometasone; oral corticosteroids, for example prednisone; bronchodilators, for example, beta-agonist bronchodilators such as albuterol, salmeterol, formoterol metaproterenol, pirbuterol, terbutaline, isoetharine, levalbuterol or salmetrol; leukotriene antagonists; for example Singulair®, i.e., montelukast sodium; and antihistamines, including for example, cetirizine, i.e., Zyrtec®, fexofenadine, i.e., Allegra®, loratadine, i.e., Claritin®, desloratadine, i.e., Clarinex®, promethazine, alimemazine, dexchlorpheniramine, brompheniramine, buclizine, carbinoxamine and doxylamine.

Upon combining the ketolide and antihistamine to treat acute exacerbations of asthma in a patient, the foregoing compounds can be present in combined pharmaceutically effective amounts to produce additive or synergistic effects, wherein each can be present in a clinical or sub clinical pharmaceutically effective amount to produce the additive or synergistic effects. As used herein, the term “additive effect” describes the combined effect of two, or more, pharmaceutically active agents that is equal to the sum of the effect of each agent given alone. The term “synergistic effect” is one in which the combined effect of two, or more, pharmaceutically active agents is greater than the sum of the effect of each agent given alone.

The drug combinations of the present invention can be provided to a patient either in separate pharmaceutically acceptable formulations administered simultaneously or sequentially, containing more than one therapeutic agent, or by an assortment of single agent and multiple agent formulations. However administered, these drug combinations form a pharmaceutically effective amount of components.

The percent reduction in symptom severity from baseline in patients in accordance with the present invention is from about 25% to about 100%. More particularly, the present invention results in at least a 50% reduction in symptom severity from baseline in patients.

The change in FEV₁ from baseline to the end of ten days' treatment seen with telithromycin in accordance with the present invention is greater than about 0.3 L. More particularly, the present invention results in a change in FEV₁ from baseline of about 0.6 L.

Experimental

The invention is further described and illustrated by the following non-limiting examples.

### EXAMPLE 1

Patients are centrally randomized (1:1) using computer-generated codes to receive blinded treatment with either oral telithromycin 800 mg once daily (two 400 mg capsules) or placebo (two capsules identical to those containing active treatment) for ten days using a proprietary Interactive Voice Recognition System to balance the treatment assignments within each study center. The first clinic visit (Visit 1), occurring within 24 hours of initial presentation is considered to be the study baseline. At baseline, patients are randomized to telithromycin or placebo. Telephone contact is made at 24-72 hours post-randomization to review concomitant medications taken and adverse events. Visit 2 is at the end of treatment (Days 11-14), Visit 3 is a post-treatment visit (Day 28 [±3 days]), and Visit 4 is the final visit (Day 42 [±3 days]). At each visit, patients undergo respiratory examinations, pulmonary function tests, health outcome assessments, and have a review of adverse events. Vital signs and concomitant medications are also recorded. Spontaneous or induced sputum samples nasopharyngeal swabs, and serum for serology for atypical pathogens are collected at Visits 1 and 3. Patients receiving at least one dose of study treatment and having one primary efficacy endpoint value are included in the intention to treat (ITT) population.

Methods

A. Efficacy, Asthma Diary Symptom Scores

Asthma symptoms are measured using a modified version of a previously published diary card symptom score in which patients rate the frequency and severity of symptoms on a 7-point Likert scale. [see Santanello N. C., Barber B. L., Reiss T. F, Friedman B. S., Juniper E. F., Zhang J., Measurement Characteristics of Two Asthma Symptom Diary Scales for Use in Clinical Trials. Eur. Respir. J. 1997; 10:646-651]. Daily patient diaries are also used to record asthma symptoms, study treatment dosing, albuterol use, and other concomitant medications. Patient's home PEF values are also recorded in triplicate twice daily.

B. Efficacy, Pulmonary Function Assessments

PEF in liter/min, FEV₁ in liters, FVC in liters, and FEF_{25-75%} in liters/sec are recorded at clinic visits and are performed according to American Thoracic Society (ATS) standards. [see American Thoracic Society. Standardization of Spirometry. Am. J. Respir. Crit. Care Med. 1995; 152: 1107-1136].

C. Efficacy, Detection of C. Pneumoniae and M. Pneumoniae

Spontaneous or induced sputum samples and nasopharyngeal swabs are obtained prior to the initiation of study treatment at Visit 1, and at Visit 3. Specimens are collected and transported by standard methods and are tested for C. pneumoniae and M. pneumoniae by polymerase chain reaction (PCR) and culture in a microbiology laboratory (G. R. Micro, London, UK). Culture and PCR are as recommended by the CDC, [see Tong C. Y., Sillis M. Detection of Chlamydia pneumoniae and Chlamydia psittaci Sputum Samples by PCR. J. Clin. Pathol. 1993; 46:313-317; see also, Dowell S. F., Peeling R. W., Boman J., et al. Standardizing Chlamydia pneumoniae Assays: Recommendations from the Centers for Disease Control arid Prevention (USA) and the Laboratory Centre for Disease Control (Canada). Clin. Infect. Dis. 2001; 33: 492-503; and see also Kong F., Gordon S., Gilbert G. L. Rapid-Cycle PCR for Detection and Typing of Mycoplasma pneumoniae in Clinical Specimens. J. Clin. Microbiol. 2000; 38, No. 11: 4256-4259], which included nested and Taqman™ assays, and assays to detect PCR inhibition and human DNA.

Acute and convalescent serum samples are also obtained for determination of titers of antibodies to M. pneumoniae and C. pneumoniae. IgM, IgG, and IgA antibodies against C. pneumoniae are detected by both microimmunofluorescence (MIF; Focus Technologies, Cypress, Calif., USA) and the Medac C. pneumoniae sandwich-enzyme-linked immunosorbent assay (ELISA; Medac, Hamburg, Germany). Serologic diagnosis of M. pneumoniae infection was performed using particle agglutination titers (Serodia-Myco II, Fujirebio Inc., Japan) and IgM ELISA (Serion, Germany). C. pneumoniae and M. pneumoniae infection is diagnosed by the presence of IgM serum antibodies, and/or a four-fold rise between baseline and convalescent samples in IgG (C. pneumoniae) or particle agglutination titer (M. pneumoniae), and/or a positive sputum or nasopharyngeal PCR, or culture.

D. Tolerability and Safety Assessments

Clinical safety is assessed in all patients by adverse-event recording and standard monitoring. Patients who receive at least one dose of steady medication and at least one safety assessment during treatment are considered evaluable for safety. All spontaneously reported adverse events and those identified by investigator observation are recorded and evaluated in terms of severity and causality.

E. Statistical Analysis

The endpoint selected for the power calculation is daytime symptom score. Assuming that the standard deviation of the mean daytime symptom score is 1.4, [see Altman L. C., Munk Z., Seltzer J., et al. A Placebo-controlled, Dose-ranging Study of Montelukast, a Cysteinyl Leukotriene-receptor Antagonist. J. Allergy Clin. Immunol. 1998; 102:50-56] a sample of 120 patients per treatment group provides 80% power to detect a 0.51 point difference between groups in decrease from baseline to end of treatment in daytime symptom score at the 0.05 significance level. This represents a 20% difference between groups in decrease from a presumed baseline score of 2.56. [see Altman L. C., Munk Z., Seltzer J., et al. A Placebo-controlled, Dose-ranging Study of Montelukast, a Cysteinyl Leukotriene-receptor Antagonist. J. Allergy Clin. Immunol. 1998; 102:50-56].

Efficacy endpoints are analyzed using an analysis of covariance (ANCOVA) model using factors for treatment, study center (investigator), treatment-by-center interaction, and baseline as covariates. Longitudinal analyses are based on the average over the 6-week study period. The treatment group means and the between-group differences are estimated using the ANCOVA model and between-group tests are used to compare telithromycin with placebo. The primary efficacy assessment time point is Visit 2 (end of treatment; Days 11-14).

Analyses of change from baseline to the end of treatment in FEV₁, FEV₁% predicted, FVC, and FEF_{25-75%} revealed a significant qualitative treatment-by-center interaction with no definitive underlying cause. In the models used, treatment effect is estimated while adjusting for the factor of center and for baseline values as covariates. This adjusted model accounts for the difference in the number of patients enrolled at each center. All mean data for efficacy outcomes are presented as least square (LS) means as they result from this adjusted model.

Improvements in asthma symptoms assessed by the patients during the ten days of study treatment are analyzed using analysis of variance (ANOVA)/ANCOVA. Time to 50% reduction of asthma symptoms is summarized for treatment groups using a Kaplan-Meier procedure.

Results

The improvements from baseline to the end of ten days' treatment seen with telithromycin (PEF, 115.8 L/min; FEV₁, 0.63 L; FVC, 0.58 L; FEF_{25-75%}, 0.85 L/sec) given in addition standard treatment, are similar to or greater than those observed with standard treatment plus placebo. Standard treatment included bronchodilators, and inhaled or oral corticosteroids in the studies discussed herein. In particular, adults with acute exacerbations of pre-existing asthma treated with telithromycin showed significantly greater improvements over placebo in asthma symptoms, time to 50% symptom recovery, symptom-free days, and four different assessments of lung function (FEV₁, PEF, FVC, FEF_{25-75%}). The differences between treatment groups in improvements in FEV₁ and symptoms are clinically important magnitude and all benefits are achieved on top of ‘usual care’ given for asthma exacerbations, including a standardized regimen of oral prednisone if considered necessary. Although the study focused primarily on the efficacy of telithromycin in the early period (first ten days) after an exacerbation, a significant improvement in symptoms persisted throughout the 6-week study period. Telithromycin was well tolerated, with a safety profile consistent with precious reports. [see Carbon C., A Pooled Analysis of Telithromycin in the Treatment of Community-acquired Respiratory Tract Infections in Adults. Infection 2003; 31:308-317].

Of the 278 patients who were enrolled in the study, 255 were included in the ITT population. The baseline demographics and disease characteristics at baseline were well balanced between the two treatment groups (Table 2), as was baseline pulmonary function (telithromycin versus placebo; FEV_{1%} predicted, 67.5% versus 66.9%; PEF % predicted, 53.5% versus 56.9%) (Table 2). During the immediate post-randomization treatment period, the two treatment groups were also well matched with respect to investigator-prescribed standardized oral corticosteroid and inhaled corticosteroid (telithromycin versus placebo; oral corticosteroids 34.1% versus 32.6% of patients; inhaled corticosteroids 83.3% versus 83.7% of patients).

A. Efficacy Results, Asthma Diary Symptom Scores

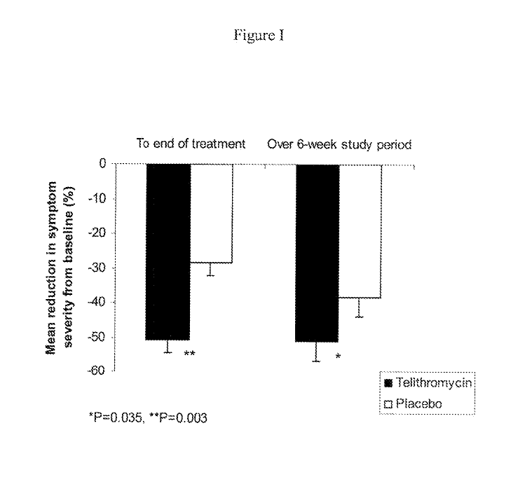

Patients in the telithromycin-treated group had significantly greater improvement in asthma symptoms compared with those in the placebo group (FIG. 1). The mean percent reduction in symptom severity from baseline to the end of treatment was −51% in the telithromycin group and −29% with placebo (mean difference between treatments −22% [95% confidence interval (CI): −36.64, −7.90%]; ) P=0.003) (FIG. 1). In addition to the greater symptomatic improvements seen during the treatment phase, patients receiving telithromycin also had significant benefit during the entire 6-week observation period of the study, with a mean change in symptom score from baseline over the study period of −51% for the telithromycin group and −38% for the placebo group (mean difference between treatments −13% [95% CI: −24.75, −0.89%]; P=0.035) (FIG. 1).

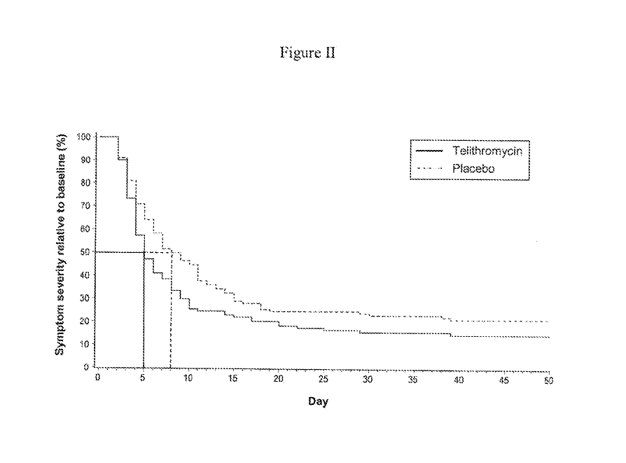

The median time to a 50% reduction in symptoms was significantly shorter in patients treated with telithromycin compared with those receiving placebo (5 days versus 8 days; P=0.031) (FIG. 2), and the proportion of patients with symptom-free days during the treatment period was significantly greater in the telithromycin group than in the placebo group (16% versus 8%; P=0.006).

B. Efficacy Results, Pulmonary Function Assessments

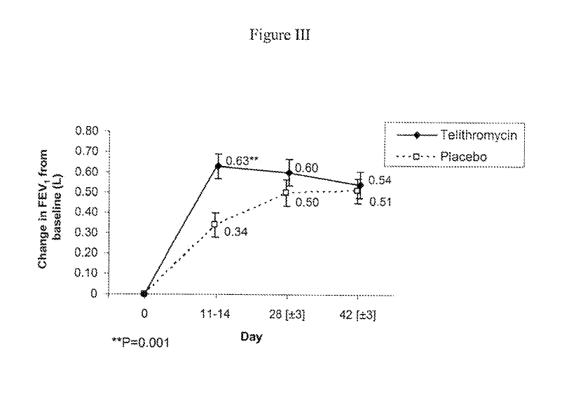

Patients treated with telithromycin had significantly greater improvements from baseline to the end of treatment compared with placebo-treated patients for all of the PFTs (FEV₁, PEF, FVC, FEF₂₅₋₇₅) performed at the clinic visits (Table 1). The improvement in FEV₁ from baseline to Visit 2 in the telithromycin group represented a mean improvement of 0.63 L versus 0.34 L in the placebo group (mean difference between treatments 0.29 L [95%) CI: 0.12, 0.46 L]; P=0.001) (FIG. 3).

TABLE 1
Mean Change from Baseline to End of Treatment for Each
Treatment Group in In-Clinic Pulmonary Function Tests

| Mean Change | | | | |
|---|---|---|---|---|
| from Baseline | Telithromycin | | Difference | |
| to End of | (800 mg/day) | Placebo | Between | Signifi- |
| Treatment | (N = 126) | (N = 129) | Treatments | cance |
| FEV₁, L | 0.63 | 0.34 | 0.29 | P = 0.001 |
| PEF, L/min | 115.8 | 88.9 | 26.9 | P = 0.036 |
| FVC, L | 0.58 | 0.31 | 0.27 | P = 0.006 |
| FEF_{25-75%}, | 0.85 | 0.45 | 0.40 | P = 0.004 |
| L/sec | | | | |

Morning home PEF measurements performed by the patients and recorded on their diary cards also showed a greater improvement in PEF from baseline over the treatment period in the telithromycin group (mean 78.3 L/min) compared with the placebo group (mean 66.8 L/min).

C. Efficacy Results, Detection of C. Pneumoniae and M. Pneumoniae

Sixty-one percent of patients met at least one of the criteria for C. pneumoniae and/or M. pneumoniae infection at baseline (Table 1). In a subgroup analysis, the magnitude of improvement in FEV₁ following telithromycin treatment was similar in the two subgroups with and without evidence of C. pneumoniae and/or M. pneumoniae infection at baseline. However, the difference between treatment groups in improvement was only statistically significant in the subgroup of patients who were diagnosed as having an infection at baseline [N=131 (61 telithromycin versus 70 placebo); mean 0.67 L versus 0.38 L; difference between treatments 0.29 L (95% CI: 0.11, 0.47 L); P=0.002]and not in patients without evidence of infection [N=86 (44 telithromycin versus 42 placebo); mean 0.65 L versus 0.47 L; difference between treatments 0.18 L (95% CI: −0.17, 0.52 L); P=0.309)9]. No differences in treatment effect, however, on symptoms were observed between infection-positive and -negative subgroups, with neither subgroup showing a statistically significant treatment effect.

D. Tolerability and Safety Assessment Results

A total of 263 patients were evaluable for safety (132 in the telithromycin group and 131 in the placebo group). The frequency of TEAEs was similar in the two groups (telithromycin, 38.6% versus placebo, 39.7%) and the majority of TEAEs were mild to moderate. TEAEs were considered to be possibly related to treatment in 44 patients (telithromycin, N=27 [20.5]%; placebo, N=27 [13.0%]). In both treatment groups, the most common adverse events that were possibly related to treatment were gastrointestinal disorders. In particular, diarrhea (telithromycin, 9.8%; placebo, 3.8%). (Of the six serious adverse events reported during the study, none was considered to be treatment-related.

TABLE 2
Baseline Demographics, Asthma and Smoking History, Bacteriologic
Status, and Pulmonary Function Tests (All Enrolled Patients)

| +HL, | Administration of | |
|---|---|---|
| | Telithromycin | Administration of |
| | (800 mg/day) | Placebo |
| 1 | (N = 140) | (N = 138) |

Demographics

| | | | | |
|---|---|---|---|---|
| Mean age (range), years ^{a} | 39.5 | (17-64) | 39.6 | (17-68) |
| Ethnic origin, N (%) ^{a} | | | | |
| White | 112 | (88.9) | 122 | (94.6) |
| Black | 3 | (2.4) | 1 | (0.8) |
| Asian/Oriental | 5 | (4.0) | 3 | (2.3) |
| Hispanic | 1 | (0.8) | 0 | (0.0) |
| Other | 5 | (4.0) | 3 | (2.3) |
| Gender, N (%) ^{a} | | | | |
| Male | 39 | (31.0) | 50 | (38.8) |
| Female | 87 | (69.0) | 79 | (61.2) |

Asthma history

| | | | | |
|---|---|---|---|---|
| Median time since asthma | 13.3 | (1-46) | 13.1 | (1-51) |
| diagnosis, years (range) | | | | |
| No. of acute exacerbations | | | | |
| in the past year, N (%) | | | | |
| 0 | 34 | (24.3) | 28 | (20.3) |
| 1 | 43 | (30.7) | 33 | (23.9) |
| 2-3 | 45 | (32.1) | 48 | (34.8) |
| >3 | 18 | (12.9) | 29 | (21.0) |

Smoking history

| | | | | |
|---|---|---|---|---|
| Smoking status, N (%) | | | | |
| Current smoker | 22 | (15.7) | 24 | (17.4) |
| Former smoker | 33 | (23.6) | 31 | (22.5) |
| Never smoked | 85 | (60.7) | 83 | (60.1) |
| Tobacco consumption, mean pack- | 2.0 | (3.2) | 2.3 | (6.3) |
| years (SD) | | | | |

Bacteriologic status, N (%) ^{b}

| | | | | |
|---|---|---|---|---|
| C. pneumoniae positive and | 4 | (3.7) | 1 | (0.9) |
| M. pneumoniae positive | | | | |
| C. pneumoniae negative and | 4 | (3.7) | 4 | (3.5) |
| M. pneumoniae positive | | | | |
| C. pneumoniae positive and | 56 | (51.4) | 67 | (58.8) |
| M. pneumoniae negative | | | | |
| C. pneumoniae negative and | 45 | (41.3) | 42 | (36.8) |
| M. pneumoniae negative | | | | |

Pulmonary function tests, mean
(SD) ^{a}

| | | | | |
|---|---|---|---|---|
| FEV₁, L | 2.18 | (0.71) | 2.30 | (0.81) |
| FEV₁% predicted, % | 67.5 | (21.9) | 66.9 | (19.7) |
| PEF L/min | 276.2 | (93.8) | 302.8 | (114.6) |
| PEF % predicted, % | 53.5 | (16.9) | 56.9 | (19.0) |
| FVC, L | 3.00 | (0.84) | 3.24 | (1.04) |
| FEF_{25-75%}, L/sec | 1.84 | (1.10) | 2.03 | (1.21) |

^{a} Intention-to-treat population (telithromycin n = 126, placebo n = 129, total n = 255).
^{b} Microbiologically evaluable population (telithromycin n = 109, placebo n = 114, total n = 223).

Discussion

This large, placebo-controlled study is the first to respond to the critical need, highlighted by current asthma treatment guidelines [see pages 63-70 of National Asthma Education and Prevention Program (NAEPP). NAEPP Expert Panel Report. Guidelines for the Diagnosis and Management of Asthma, Update on Selected Topics 2002, accessed Nov. 15, 2005, at http://www.nhlbi.nih.gov/guidelines/asthma/asthmafullrpt.pdf], i.e., that a high-quality assessment was needed to determine whether there is evidence regarding the role of antibiotic therapy in the treatment of acute exacerbations of asthma. This study demonstrates statistically significant and clinically substantial benefits associated with this antibiotic treatment in acute exacerbations of asthma.

In particular, the data shows clinically relevant benefits in all key efficacy parameters in adult patients with acute asthma exacerbations. It is believed that the established effectiveness arises from the ketolides being bactericidal against C. pneumoniae and M. pneumoniae whereas macrolides are bacteriostatic [see Gustafsson I., Hjelm F., Cars O., In Vitro Pharmacodynamics of the New Ketolides HMR 3004 and HMR 3647 (Telithromycin) Against Chlamydia pneumoniae. Antimicrob. Agents Chemother. 2000; 44:1846-1849] and that may exert greater immunomodulatory effects than the macrolides.

From the in vitro and in vivo activity of telithromycin discussed herein it is believed that the antibacterial activity of telithromycin may be responsible at least in part for the treatment effects seen with the present invention. This is supported by telithromycin data showing that 61& had serologic, culture, or PCR evidence of C. pneumoniae and/or M. pneumoniae infection and perhaps by the observation that the effect of telithromycin on FEV₁ was statistically significant in patients with documented infection at baseline and not in those patients without evidence of infection. However, the interpretation of the results of this study with respect to C. pneumoniae and M. pneumoniae infection is problematic due to the lack of standardized laboratory tests to accurately diagnose infection status for these organisms. It is generally recognized that standardized laboratory tests to accurately diagnose infection status for C. pneumoniae and M. pneumoniae are lacking in the relevant field. [see Dowell S. F., Peeling R. W., Boman J., et al. Standardizing Chlamydia pneumoniae Assays: Recommendations from the Centers for Disease Control and Prevention (USA) and the Laboratory Centre for Disease Control (Canada). Clin. Infect. Dis. 2001; 33: 492-503; and see also Daxboeck F., Krause R., Wenisch C. Laboratory Diagnosis of Mycoplasma pneumoniae Infection. Clin. Microbiol. Infect. 2003; 9, No. 4: 263-273]. Therefore, it is not surprising that the interpretation of results herein may also be problematic. Further, the magnitudes of treatment effects of this study were similar in the infection-positive and -negative groups. The lack of statistical significance, however, in the latter may simply relate to a smaller number of patients in that particular group.

We claim:

1. A method of treating a patient suffering from, or subject to, acute asthma exacerbations comprising administering to the patient a pharmaceutically effective amount of a ketolide.

2. The method according to claim 1 wherein the ketolide is telithromycin.

3. The method according to claim 1 wherein the administering is oral, intravenous or inhalational.

4. A method for treating according to claim 1 further comprising administering a pharmaceutically effective amount of at least one an additional therapeutic agent selected from the group consisting of an inhaled corticosteroid, oral corticosteroid, bronchodilator, leukotriene antagonist and an antihistamine.

5. The method according to claim 4 wherein the inhaled corticosteroid is selected from the group consisting of beclomethasone, budesonide, fluticasone and mometasone.

6. The method according to claim 4 wherein the oral corticosteroid is prednisone.

7. The method according to claim 4 wherein the bronchodilator is a beta-agonist.

8. The method according to claim 7 wherein the beta-agonist bronchodilator is selected from the group consisting of albuterol, salmeterol, formoterol, metaproterenol, pirbuterol, terbutaline, isoetharine, levalbuterol and salmetrol.

9. The method according to claim 4 wherein the leukotriene antagonist is montelukast sodium.

10. The method according to claim 4 wherein the antihistamine is selected from the group consisting of cetirizine, fexofenadine, loratadine, desloratadine, promethazine, alimemazine, dexchlorpheniramine, brompheniramine, buclizine, carbinoxamine and doxylamine.

11. The method according to claim 1 wherein the treating is effected in part through bactericidal activity of the ketolide.

12. The method according to claim 11 wherein the bactericidal activity is against C. pneumoniae and M. pneumoniae.

13. The method according to claim 1 wherein the treating is effected in part through immunomodulatory activity of the ketolide.

14. The method according to claim 1 wherein the treating is effected in part through anti-inflammatory activity of the ketolide.
